(19) United States
(12) Patent Application Publication
(10) Pub. No.: US 2006/0051421 A1
(43) Pub. Date: Mar. 9, 2006

(54) Stable pharmaceutical formulations of benzimidazole compounds

(76) Inventors: Nava Shterman, Petach Tikva (IL); Simona Di Capua, Kfar Saba (IL); Benny Moshe, Kfar Saba (IL); Esther Itah, Netanya (IL)

Correspondence Address: KENYON & KENYON, ONE BROADWAY, NEW YORK, NY, 10004, US

(21) Appl. No.: 11/153,954

(22) Filed: Jun. 15, 2005

Related U.S. Application Data
(60) Provisional application No. 60/580,273, filed on Jun. 15, 2004.
(60) Provisional application No. 60/588,233, filed on Jul. 14, 2004.
(60) Provisional application No. 60/591,784, filed on Jul. 27, 2004.

(51) Int. Cl. A61K 31/4439; A61K 9/24

(52) U.S. Cl. 424/472; 514/338

(57) ABSTRACT

Provided are stable pharmaceutical formulations of benzimidazole compounds, particularly esomeprazole magnesium, and processes for their preparation.

### CROSS REFERENCE TO RELATED APPLICATIONS

This application claims the benefit of U.S. provisional application Ser. No. 60/580,273, filed Jun. 15, 2004, U.S. provisional application Ser. No. 60/588,233, filed Jul. 14, 2004, and U.S. provisional application Ser. No. 60/591,784 filed Jul. 27, 2004, all of which are incorporated herein by reference.

### FIELD OF THE INVENTION

The present invention relates to a pharmaceutical formulation comprising a benzimidazole compound. In particular, the present invention relates to a stable pharmaceutical formulation comprising esomeprazole magnesium and a method of its preparation.

### BACKGROUND OF THE INVENTION

Esomeprazole is a substituted benzimidazole compound. It is chemically known as bis (5-methoxy-2-[2(S)-[4-methoxy-3,5-dimethyl-2-pryidinyl)methyl]sulfinyl]-1H-benzimidazole-1-yl, having the following structure: 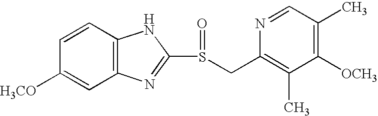
It is an effective gastric acid secretion inhibitor and used for the treatment of gastric and duodenal ulcer, severe erosive esophagitis, Zolinger-Ellison syndrome and H-pylori eradication. Esomeprazole magnesium is often used in combination with an antibiotic such as clarithromycin or amoxicillin. Other substituted benzimidazole compounds include lansoprazole, omeprazole, pantoprazole, and rabeprazole. While omeprazole is a mixture of S- and R-isomers, esomeprazole represents the S-isomer of omeprazole. Lansoprazole has the following structure: 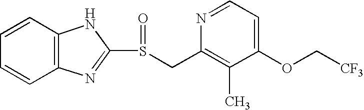
Pantoprazole has the following structure: 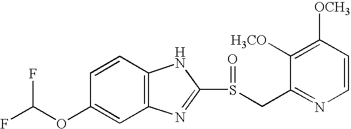
Rabeprazole has the following structure:

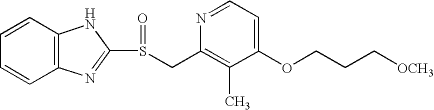

It has been reported that benzimidazole compounds have poor stability when exposed to acidic conditions. The stability is reported to decrease with decreasing pH. For example, the half-life of an aqueous esomeprazole composition at a pH of 6.5 is reported to be of the order of about 18 hours whereas the half-life at a pH of 4 is reported to be of the order of about 10 minutes. The stability of benzimidazole compounds also has been reported to decrease with high heat and moisture.

Benzimidazole compounds have been reported to be acid labile. As such, they are generally designed as enteric coated dosage forms in order to avoid degradation of the active pharmaceutical ingredient (API) at the low pH found in the stomach. However, because enteric coatings are generally comprised of acidic compounds, direct covering of the benzimidazole compounds with these types of coatings has been reported to cause degradation and decomposition of the active pharmaceutical ingredient, causing the active pharmaceutical ingredient preparation to undergo discoloration and to lose its active ingredient content over time.

Various enteric coated formulations have been developed in the art to address the lack of stability of benzimidazole compounds. U.S. Pat. No. 5,690,960 for example claims “a stable oral formulation comprising: a core containing a magnesium salt of omeprazole said salt having more than 70% crystallinity as determined by x-ray powder diffraction; a subcoating layer; and an enteric coating layer, whereby the thickness of the enteric coating layer does not effect the release of omeprazole into solution at the pH predominantly present in the small intestine.”

U.S. Pat. No. 6,090,827 according to its abstract, provides “an enteric coated oral pharmaceutical formulation comprising as active ingredient a compound selected from the group of omeprazole, an alkaline salt of omeprazole, the (−)-enantiomer of omeprazole and an alkaline salt of the (−)-enantiomer of omeprazole, wherein the formulation comprises a core material of the active ingredient and optionally an alkaline reacting compound, the active ingredient is in admixture with a pharmaceutically acceptable excipient, such as for instance a binding agent, and on said core material a separating layer and an enteric coating layer. A hydroxypropyl methylcellulose (HPMC) of low viscosity with a specific cloud point is used in the manufacture of pharmaceutical formulations.”

U.S. Pat. No. 6,428,810, according to its abstract, provides “an enteric coated oral pharmaceutical formulation comprising as active ingredient a compound selected from the group of omeprazole, an alkaline salt of omeprazole, one of the single enantiomers of omeprazole and an alkaline salt of one of the single enantiomers of omeprazole, wherein the formulation comprises a core material that comprises the active ingredient and optionally an alkaline reacting compound, the active ingredient is in admixture with a pharmaceutically acceptable excipient, such as for instance a binding agent, and on said core material a separating layer and an enteric coating layer.”

U.S. Pat. No. 4,786,505, according to its abstract, provides “pharmaceutical preparation containing omeprazole together with an alkaline reacting compound or an alkaline salt of omeprazole optionally together with an alkaline compound as the core material, one or more subcoating layers comprising inert reacting compounds which are soluble or rapidly disintegrating in water, or polymeric, water soluble film forming compounds, optionally containing pH-buffering alkaline compounds and an enteric coating as well as a process for the preparation thereof and the use in the treatment of gastrointestinal diseases.”

U.S. Pat. No. 4,853,230, according to its abstract provides “pharmaceutical preparation containing an acid labile compound together with an alkaline reacting compound or an alkaline salt of an acid labile compound optionally together with an alkaline compound as the core material, one or more subcoating layers comprising inert reacting compounds which are soluble or rapidly disintegrating in water, or polymeric, water soluble film forming compounds, optionally containing pH-buffering alkaline compounds and an enteric coating as well as a process for the preparation thereof and the use in the treatment of gastrointestinal diseases.”

According to the abstract of U.S. Pat. No. 5,626,875, “stable oral pharmaceutical formulations are prepared by covering an inert nucleus with a first layer containing an acid labile benzimidazole compound, a water soluble polymer and non-alkaline reacting pharmaceutical acceptable excipients, a second isolation layer containing a water soluble polymer, pharmaceutical acceptable excipients and a final enteric coating.”

U.S. Pat. Nos. 4,786,505 and 4,853,230, mentioned above, have been subject to litigation in the United States. See Astra Aktiebolag v. Andrx Pharm., Inc., 222 F. Supp.2d 423 (S.D.N.Y 2002).

There is a continuing need to prepare a stable pharmaceutical formulation containing benzimidazole compounds, especially esomeprazole magnesium.

### SUMMARY OF THE INVENTION

The present invention provides a pharmaceutical formulation of a benzimidazole compound, comprising:
a) an inert inner core;
b) a first coating on the inner core comprised of a benzimidazole compound and an alkaline stabilizer;
c) an intermediate layer on top of the first coating devoid of an alkaline stabilizer comprised of the same or different benzimidazole compound; and
d) an outer enteric layer.
   Preferably, the inner core is made of inert non-pareil sugar spheres, MCC spheres, glass beads, coarse grade silicon dioxide particles.

Preferably, the benzimidazole compound is lansoprazole, omeprazole, pantoprazole or rabeprazole, more preferably esomeprazole. Preferably, the benzimidazole compound is a salt, such as a lithium, sodium, calcium, potassium or magnesium salt. More preferably, the benzimidazole compound is esomeprazole magnesium. More preferably, the benzimidazole compound is an amorphous (including partially amorphous) form of esomeprazole. The benzimidazole compound may exist in a hydrated state.

Preferably, the benzimidazole compound present on the first coating is in the amount of about 80% to about 95%, more preferably about 85% to about 95%, and most preferably about 90% (w/w) of the total amount of the benzimidazole compound in the formulation.

Preferably, the benzimidazole compound is layered onto the inert non-pareil core.

The intermediate layer is devoid of alkaline stabilizer and comprises the same or different benzimidazole compound. Preferably, the benzimidazole compound present in the intermediate coating is about 5% to about 20%, more preferably about 5% to about 15%, and most preferably 10% (w/w) of the total amount of the benzimidazole compound in the formulation.

Preferably, the pharmaceutical formulation of the present invention is in the form of a multi-particulate delivery system. The multi-particulate delivery system comprises a plurality of particles having:
a) an inert inner core;
b) a first coating on top of the inner core comprised of a benzimidazole compound and an alkaline stabilizer;
c) an intermediate layer on top of the first coating devoid of the alkaline stabilizer comprising the benzimidazole compound; and
d) an outer enteric layer.

Preferably, the inert core is microcrystalline cellulose or sugar sphere.

Preferably, the benzimidazole compound present in the inner core is in the amount of about 90% (w/w) of the labeled dose of benzimidazole compound in the formulation.

Preferably, the benzimidazole compound present in the intermediate layer is in the amount of about 10% (wlw) of the labeled dose of benzimidazole compound in the formulation.

The present invention provides a pharmaceutical composition comprising a benzimidazole compound that is resistant to dissolution in acidic dissolution media for about 2 hours. The stable pharmaceutical composition dissolves within 1 hour when the media is changed to alkaline buffer.

The present invention provides a process of preparing a stable pharmaceutical composition of a benzimidazole compound, comprising the steps of:
a) coating an inert inner core with a suspension comprising a benzimidazole compound and an alkaline stabilizer;
b) layering the coated inner core with an intermediate layer; and
c) layering the intermediate coating with an outer enteric layer,
wherein the intermediate layer is devoid of an alkaline stabilizer and comprises the same or different benzimidazole compound.

Preferably, the inner core is an inert sugar sphere or a microcrystalline cellulose (MCC) sphere. The first coating on the inner core comprises a benzimidazole compound and an alkaline stabilizer. Preferably, the coating is performed by layering on the inert sugar sphere/MCC sphere with a suspension comprising a binder, a benzimidazole compound and a basic inorganic salt. Preferably, the benzimidazole compound is esomeprazole magnesium. Preferably, the alkaline stabilizer is magnesium carbonate

Preferably, the intermediate layer comprises a binder, a benzimidazole compound and an anti-tackiness agent. Preferably the anti-tackiness agent is talc or magnesium stearate. Preferably, the layer is applied by coating the inner spheres with a suspension that comprises a binder, benzimidazole compound and an antitacking agent and is devoid of alkaline stabilizer.

The binder for both the first layer/coating or the intermediate layer/coating is preferably selected from the group consisting of hydroxypropyl methylcellulose, hydroxypropylcellulose, polyvinyl alcohol, polyvinyl pyrrollidone, starch, methylcellulose, ethylcellulose, carboxymethyl cellulose, sucrose solution, dextrose solution.

The enteric layer may be applied from an aqueous suspension or an organic solvent solution. Preferably, the outer enteric layer is layered by an aqueous suspension or solvent solution that comprises talc extra fine, titanium dioxide, triethyl citrate and methacrylic acid copolymer.

The present invention provides a stable pharmaceutical formulation of a benzimidazole compound, comprising:
a) an inner core comprised of a benzimidazole compound and an alkaline stabilizer;
b) an intermediate layer on said inner core devoid of the alkaline stabilizer comprising the same or different benzimidazole compound; and
c) an outer enteric layer.

### BRIEF DESCRIPTION OF THE FIGURES

FIG. 1 provides a dissolution profile for the oral dosage forms prepared in all the actual (non-prophetic) examples.

### DETAILED DESCRIPTION OF THE INVENTION

The present invention provides for a formulation of a benzimidazole compound where a benzimidazole-containing layer is used to separate the benzimidazole compound from an acidic enteric coating. Such formulation is stable despite lack of presence of an alkaline layer or a physical barrier between the benzimidazole compound and the enteric coating.

In one embodiment, the stable pharmaceutical formulation of the present invention comprises: a) an inert inner core b) a first coating on the inner core comprised of a benzimidazole compound and an alkaline stabilizer; c) an intermediate layer on said first coating devoid of an alkaline stabilizer comprised of the same or different benzimidazole compound; and d) an outer enteric layer.

Preferably, the benzimidazole compound is lansoprazole, omeprazole, pantoprazole, rabeprazole or esomeprazole, or a salt thereof. More preferably, the benzimidazole compound is esomeprazole magnesium. The benzimidazole compound may be present in amorphous or crystalline state. The amorphous form may be partially amorphous and contain up to 20% by weight crystallinity. Preferably, the benzimidazole compound is esomeprazole magnesium amorphous. The benzimidazole compound, particularly esomeprazole magnesium, may be hydrated.

The benzimidazole compound of the present invention preferably has the following structure: 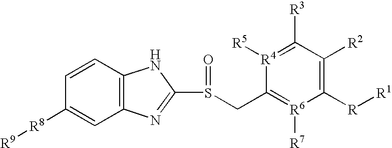
wherein R⁴ and R⁶ are independently selected from carbon and nitrogen,
R and R⁸ are independently hydrogen or oxygen,
R¹, R², R³, R⁵, R⁷, R⁹ are independently selected from hydrogen, halogen, methyl, ethyl, propyl, methoxy, ethoxy, acetate, ethyl acetate, C₁-C₈ ether, optionally substituted with a halogen. Specific examples of such compounds are lansoprazole, pantoprazole, pariprazole, laminoprazole, omeprazole and esomeprazole. More preferably, the benzimidazole compound is esomeprazole magnesium (an S-isomer of omeprazole).

An inert core is layered with the benzimidazole compound. In a preferred embodiment, the core includes a non-pareil core, to which the benzimidazole compound and the alkaline stabilizers are added as a layer. Examples of inert non-pareil spheres include sugar spheres, microcrystalline cellulose spheres (MCC), glass beads and coarse grade silicon dioxide cores. The inert sphere is preferably about 30% to about 90% (w/w) of the drug layered core. The inert sphere preferably has a mean diameter of about 250 to about 1,200 microns, more preferably a mean diameter of about 400 to about 700 microns.

The first coating on the inner core of the pharmaceutical dosage form of the present invention contains preferably of about 80% to about 95%, more preferably of about 85% to about 95%, and most preferably 90% of the total benzimidazole compound. The first coating also contains an alkaline stabilizer.

The term alkaline stabilizer refers to a pharmaceutically acceptable alkaline, or basic substance. According to U.S. Pat. No. 6,103,281, examples of such alkaline stabilizers include organic buffering compounds such as trometamine (i.e. Tris-buffer), N-amino sugars such as N-methyl-D-glucamine (i.e. Meglumine), N-ethyl-D-glucamine (i.e. Eglumine), alkali salts of citric acid, tartaric acid, alkali metal phosphates, silicates or carbonates, sodium, potassium, magnesium, calcium or aluminum hydroxides and organic amines such as ethylamine, dicyclohexylamine or triethanolamine, or alkaline ammonium salts.

Preferred alkaline stabilizers are inorganic basic salts such as magnesium carbonate, magnesium oxide, magnesium hydroxide, magnesium silicate aluminate, magnesium silicate, calcium carbonate, calcium hydroxide, sodium carbonate, sodium hydrogen carbonate. Most preferred alkaline stabilizers are magnesium carbonate, magnesium oxide, calcium carbonate and sodium carbonate.

The coating contains a benzimidazole compound or a combination of benzimidazole compounds. The benzimidazole compounds may be present in their salt forms.

The core is coated with a suspension comprising a benzimidazole compound and an alkaline stabilizer. The coating process is exemplified by a “Wurster” type column-equipped fluidized bed apparatus (i.e., Bottom spray technique). The drug layered core preferably comprises: a) about 80% to about 95% (w/w) of the labeled dose of a benzimidazole compound in the formulation; b) about 2% to about 30% (w/w) of the drug layered core of a binder polymer; c) and about 2% to about 30% (w/w) of an alkaline stabilizing agent.

The benzimidazole compound, binder, and alkaline stabilizer are combined with water to obtain an aqueous suspension, which is then applied to the core. The binder polymer is preferably one or more, or mixtures thereof, of hydroxypropyl methylcellulose, hydroxypropylcellulose, or polyvinyl alcohol.

An intermediate layer is then placed on the coated core. Preferably, the intermediate layer's is about 30% to about 70% (w/w) of the drug layered core.

The intermediate layer is devoid of an alkaline stabilizer, but contains a benzimidazole compound. Preferably, the benzimidazole compound in the intermediate layer is present in the amount of about 5% to about 20%, more preferably about 5% to about 15%, and most preferably about 10% of the labeled dose.

The intermediate layer may contain an inert polymer. The inert polymer may act as a binding agent. The binding agent is exemplified by hydroxypropyl methylcellulose, hydroxypropyl cellulose, polyvinyl alcohol and ethylcellulose. Additional binding agents may include, but are not limited to, polyvinyl pyrrollidone, starch, methylcellulose, carboxymethyl cellulose, sucrose solution, dextrose solution. The anti-tackiness agents include talc, glyceryl monostearate, silicon dioxide and metallic stearates such as magnesium stearate.

The benzimidazole compound and the binding agent is preferably sprayed from an alcoholic suspension, or a mixture of water and an alcohol. The suspension may be prepared by combining the benzimidazole compound, the binding agent and an anti-tackiness agent in water, a C₁ to C₄ alcohol, or mixtures thereof. A preferred alcohol is ethanol.

Preferably, the binding agent is about 10% to about 70% (w/w) of the intermediate layer. More preferably, the binding agent is about 20% to about 60% (w/w) of the intermediate layer weight. Preferably, the anti-tackiness agent is about 10% to about 85% (w/w) of the intermediate layer weight. More preferably, the anti-tackiness agent is about 40% to about 70% (w/w) of the intermediate layer weight.

An enteric coating is then placed on the intermediate layer. An enteric layer/coating usually includes a polymer with enteric properties such as methacrylic acid copolymer, hydroxypropyl methylcellulose phtalate or hydroxypropyl methylcellulose acetate succinate. Additional enteric polymers include cellulose acetate phthalate, polyvinyl acetate phthalate, cellulose acetate trimellitate, shellac or zein.

The enteric coating may be prepared by coating the finished product with a solution or a homogeneous dispersion of the enteric polymer in water, an organic solvent or mixtures thereof. The solution or dispersion may have an anti-tackiness agent, plasticizer, pigments, etc.

In a preferred embodiment, the enteric coating is applied from a solution of the enteric polymer in a mixture of organic solvents. The solution may be prepared in a polar organic solvent such as C₁ to C₄ alcohol, C₃ to C₇ esters, ethers and ketones. A preferred solvent mixture is that of acetone and isopropyl alcohol, preferably from about a 5:1 to about 1:2 ratio (w/w), more preferably about a 3:2 mixture.

The enteric layer may include other ingredients: an anti-tackiness agent such as talc or glyceryl monostearate; a plasticizer such as triethylcitrate or polyethylene glycol; and pigments such as titanium dioxide or ferric oxides.

Additional plasticizers may include, but not limited to, acetyl triethyl citrate, acetyl tributyl citrate, acetylated monoglycerides, glycerin, triacetin, propylene glycol, phthalate esters (e.g., diethyl phthalate, dibutyl phthalate), castor oil, sorbitol and dibutyl seccate.

Preferably, the enteric layer is about 10% to about 50% (w/w) of the final formulation. Preferably, the enteric polymer is about 45% to about 85% (w/w) of the enteric layer weight.

Preferably, the anti-tackiness agent is about 2 to about 40% (w/w) of the enteric layer weight. Preferably, the plasticizer is about 2 to about 15% (w/w) of the enteric layer weight. Preferably, the pigment is about 0.5 to about 10% (w/w) of the enteric layer weight.

The enteric coating solution or dispersion is preferably sprayed on the multiply coated inner core. The enteric coated product may be dried. The enteric coated product may then be separated based on ideal size, for example by sifting through a multiple mesh screen. Particles having a mean diameter of about 400 to about 1200 microns are preferred.

In another embodiment, instead of an inert core, a core with a benzimidazole compound and an alkaline stabilizer is used. The core with the benzimidazole compound and the alkaline stabilizer may be prepared for example by extrusion and spheronization or as a tablet or mini-tablet core. In this embodiment, the powder mass of the benzimidazole compound, an alkaline stabilizer and preferably microcrystalline cellulose are mixed with water or solvent to obtain a suitable consistency, followed by extrusion from a screen with a suitable size, such as about a 0.5 mm to about a 2 mm screen. The extrudate is formed into pellets and then dried in a fluidized bed drier. See e.g. U.S. Pat. No. 6,013,281. In another embodiment, the benzimidazole compound and the alkaline stabilizer are mixed and wet granulated, followed by drying of the wet granules. The granules may then be sieved, and other excipients added for compression into a core. See e.g. U.S. Pat. No. 6,013,281. The core so produced is then coated with an intermediate layer and an outer enteric layer as described above.

The pharmaceutical formulation of the present invention can be further coated with one or more seal coatings, film coatings, barrier coatings, compression coatings, fast disintegrating coatings, or enzyme degradable coatings. Multiple coatings, including multiple enteric coatings, can be applied for desired performance. Furthermore, the dosage form can be designed for intermediate release, pulsatile release, controlled release, extended release, delayed release, targeted release, synchronized release, or targeted delayed release.

For release/absorption control, solid carriers can be made of various component types and levels or thickness of coats, with or without an active ingredient. Such diverse solid carriers can be blended in a dosage form to achieve a desired performance. In addition, the dosage form release profile can be effected by a multiparticulate composition, a coated multiparticulate composition, an ion-exchange resin-based composition, an osmosis-based composition, or a biodegradable polymeric composition. The rate of release may be effected through favorable diffusion, dissolution, erosion, ion-exchange, osmosis or combinations thereof.

A multiparticulate dosage form includes a plurality of the coated particles, such as the MCC spheres. A preferred size range for the particles in such dosage form is a mean diameter of about 400 to about 1200 microns.

When formulated as a capsule, the capsule can be a hard gelatin capsule, a starch capsule, or a cellulosic capsule. Although not limited to capsules, such dosage forms can further be coated with, for example, a seal coating, an enteric coating, an extended release coating, or a targeted delayed release coating.

The formulations of the present invention may be used to treat erosive esophagitis (chronic and/or inflammation of the esophagus) and gastroesophageal reflux (heartburn). They may also be used individually or in combination with antibiotics such as clarithromycin and amoxicillin to treat duodenal (intestinal) ulcers caused by the bacteria Helicobacter pylori.

The formulations of the present invention may be preferably administered at a dose of about 5 mg to about 80 mg, more preferably about 20 mg to about 40 mg. The current dosage for esomeprazole magnesium depends on the particular conditions treated. For treating active erosive esophagitis, a patient takes about 20 or 40 mg a day for about 4 to 8 weeks. The prescribed maintenance dose for esophagitis is about 20 mg a day. The dose for gastroesophageal reflux is about 20 mg a day for 4 weeks. The does to reduce the risk of duodenal ulcer recurrence is about 40 mg esomeprazole a day for 10 days, in combination with about 1,000 mg amoxicillin twice a day for 10 days, and about 500 mg clarithromycin twice a day for 10 days.

The stability of the esomeprazole magnesium formulation of the present invention was monitored, according to the pharmaceutical industry standard, under accelerated conditions of about 30° C. and about 65% relative humidity for three months. The final preparation showed satisfactory stability at these conditions. Table 1 provides results of such stability tests. Preferably, after two months of storage under such conditions, more preferably after three months, the oral dosage form has an assay of more than about 98%, more preferably more than about 99%, and most preferably about 100% as compared to a batch of pure esomeprazole magnesium.

TABLE 1
Stability of esomeprazole magnesium capsules 40
mg at 30° C. and 65% relative humidity

| Assay % | Example 1 | Example 2 | Example 5 | Example 6 | Example 7 |
|---|---|---|---|---|---|
| Time zero | 100% | 101% | 99% | 103% | 101% |
| 3 months | 101% | 101% | 99% | 100% | 98%* |

*2 months

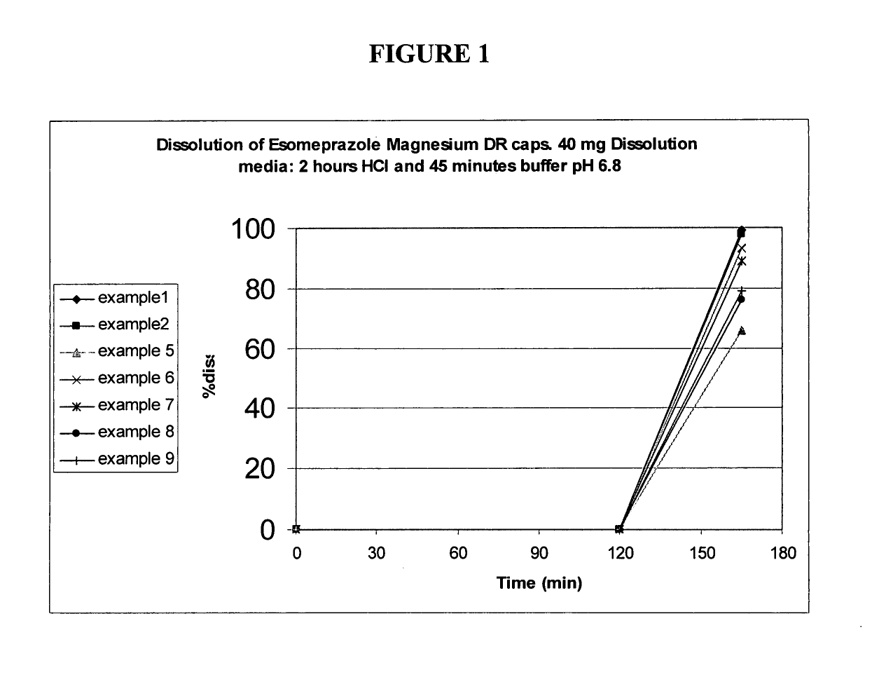

Further, the formulation of the present invention is resistant to dissolution in acidic dissolution media for at least about 2 hours, but dissolves within about 1 hour when the media is changed to an alkaline buffer. Such lack of dissolution in acidic media is beneficial because the benzimidazole compound degrades under acidic conditions. The dissolution profiles of the oral dosage forms of the present invention are illustrated in FIG. 1.

The disclosures of the cited publications are incorporated herein in their entireties by reference. It is to be understood, however, that the scope of the present invention is not to be limited to the specific embodiments described above. The invention may be practiced other than as particularly described and still be within the scope of the accompanying claims.

The formulation as per this invention may be analyzed by the following techniques:

Dissolution Method

2 Stages:
I. Acid Stage: 300 ml 0.1N HCl, 37° C., 100 rpm, 120 minutes
II. Buffer stage: 1000 ml phosphate buffer pH 6.8, 37° C., 100 rpm, 60 minutes
   HPLC Method
Column: Luna C18(2) HXY-6 μm
Column Temp: 30° C.
Mobile phase: water:acetonitrile:triethylamine 60:40:1, pH 7.0
Detector: ultraviolet at wavelength 302 μm
Injection Volume: 10 ml
Flow: 1 ml/minute
   Assay and IDD Determination (Impurity Degradation Determination)
Column: Xterra 150×4.6 um
Column Temp: 30° C.
Mobile Phase: Phosphate buffer (1.1 grams sodium phosphate dibasic anhydrous and 1.0 grams tetrabutyl ammonium hydrogen phosphate dissolved in water) and acetonitrile
Gradient program (Solvent A and Solvent B):
Solvent A=90% buffer: 10% acetonitrile
Solvent B=50% buffer:50% acetonitrile
Detection: ultraviolet wavelength at 302 μm, flow 1.5 ml/min.

### EXAMPLES

The following non-limiting examples further illustrate the invention.

### Example 1

A. Drug Layer

Drug Layer Coating Suspension

180 g of hydroxypropyl methylcellulose NF 6 cps was dispersed in 3.8 kg of purified water. 180 g magnesium carbonate was added and the solution was stirred. 240 g esomeprazole magnesium was added and stirred until a homogeneous suspension was obtained.

720 g Cellets® (microcrystalline cellulose spheres) (500-710 micron) were introduced into a fluid bed apparatus and the aforementioned suspension was sprayed onto the spheres. Then the spheres were dried, sifted through a 18 mesh screen and were replaced into the fluidized bed apparatus for further coating.

B. Intermediate Coating

Intermediate Coating Suspension

145 g of hydroxypropyl methylcellulose NF 6 cps was dispersed in 1.9 kg of purified water. 22.5 g esomeprazole magnesium was added to hydroxypropyl methylcellulose solution and stirred. 290 g of magnesium stearate were dispersed in 1.3 kg of ethanol 96%. The intermediate coating suspension was sprayed onto 1.1 kg of drug layered pellets from the previous step. The spheres were then dried, sifted through a 18 mesh screen and replaced into the fluidized bed apparatus for further coating.

C. Enteric Coating

Enteric Coating Dispersion

50.4 g of talc extra fine, 6.3 g of titanium dioxide and 12.83 g of triethyl citrate were dispersed in 600 g of purified water. The homogenized dispersion was added to 422.3 g of methacrylic acid copolymer dispersion and stirred.

The enteric coating dispersion was sprayed onto 700.9 g of spheres from the previous step. The spheres were then dried, sifted through a 16 mesh screen and filled into hard gelatin capsules.

### Example 2

A. Drug Layer

Drug Layer Coating Suspension

210 g of hydroxypropyl methylcellulose NF 6 cps was dispersed in 4.9 kg of purified water. 210 g magnesium carbonate was added and the solution was stirred. 280 g esomeprazole magnesium was added and stirred until a homogeneous suspension was obtained.

700 g Suglets® (sugar spheres) (500-600 micron) were introduced into a fluid bed apparatus and the aforementioned suspension was sprayed onto the spheres. Then the spheres were dried, sifted through a 18 mesh screen and were replaced into the fluidized bed apparatus for further coating.

B. Intermediate Coating

Intermediate Coating Suspension

156.8 g of hydroxypropyl methylcellulose NF 6 cps was dispersed in 1.9 kg of purified water. 24.8 g esomeprazole magnesium was added to hydroxypropyl methylcellulose solution and stirred.77 g of ethocel 7cps were dispersed in 1.1 kg ethanol 96%. 319 g of magnesium stearate were dispersed in 1.4 kg of ethanol 96%. All three dispersions were mixed together and stirred. The intermediate coating suspension was sprayed onto 1.1 kg of drug layered pellets from the previous step. The spheres were then dried, sifted through a 18 mesh screen and replaced into the fluidized bed apparatus for further coating.

C. Enteric Coating

Enteric Coating Dispersion

55 g of talc extra fine, 6.3 g of titanium dioxide and 13.8 g of triethyl citrate were dispersed in 900 g of purified water. The homogenized dispersion was added to 462.5 g of methacrylic acid copolymer dispersion and stirred.

The enteric coating dispersion was sprayed onto 762.5 g of spheres from the previous step. The spheres were then dried, sifted through a 16 mesh screen and filled into hard gelatin capsules.

### PROPHETIC Example 3

### (Extruded/Spheronized Inner Core)

Inner core containing benzimidazole and alkaline stabilizer

| | |
|---|---|
| Esomeprazole magnesium | 400 g |
| Microcrystalline cellulose | 750 g |
| Magnesium carbonate | 300 g |
| HPMC 6 cps | 50 g |

A. Drug Core

The inner core containing esomeprazole magnesium and magnesium carbonate as an alkaline stabilizer is prepared by extrusion/spheronization process.

The powder mixture is mixed in a high shear mixer and water or hydro-alcoholic solution is added to obtain a suitable wet mass. Extrusion is performed with the aid of an extruder apparatus fitted with 0.6 mm screen. The extrudates are spheronized with the aid of a spheronizer machine and dried in a fluidized bed dryer.

Then the spheres are dried, sifted through a 18 mesh screen and replaced into the fluidized bed apparatus for further coating.

B. Intermediate Coating

Intermediate Coating Suspension

145 g of hydroxypropyl methylcellulose NF 6 cps is dispersed in 1.9 kg of purified water. 22.5 g esomeprazole magnesium is added to the hydroxypropyl methylcellulose solution and stirred. 290 g of magnesium stearate are dispersed in 1.3 kg of ethanol 96%. The intermediate coating suspension is sprayed onto 750 g of drug layered pellets from the previous step. The spheres are then dried, sifted through an 18 mesh screen and replaced into the fluidized bed apparatus for further coating.

C. Enteric Coating

Enteric Coating Dispersion

50.4 g of talc extra fine, 6.3 g of titanium dioxide and 12.83 g of triethyl citrate are dispersed in 600 g of purified water. The homogenized dispersion is added to 422.3 g of methacrylic acid copolymer dispersion and stirred.

The enteric coating dispersion is sprayed onto 543.4 g of spheres from the previous step. The spheres are then dried, sifted through a 16 mesh screen and filled into hard gelatin capsules.

### PROPHETIC Example 4

### (Tablet Inner Core)

Inner core containing benzimidazole and alkaline stabilizer

| | |
|---|---|
| Esomeprazole magnesium | 400 g |
| Macrocrystalline cellulose | 750 g |
| Magnesium carbonate | 300 g |
| HPMC 6 cps | 50 g |

Extra granular excipients

| | |
|---|---|
| Microcrystalline cellulose | 50 g |
| Magnesium stearate | 2 g |

A. Drug Layer

The inner core containing esomeprazole magnesium and magnesium carbonate as an alkaline stabilizer is prepared by a wet granulation process.

The powder mixture is mixed in a high shear mixer and water or hydro-alcoholic solution is added to obtain a suitable granulation. The obtained granulation is dried in a fluidized bed dryer, milled through 0.6 mm screen mixed with extra-granular excipients and compressed into core tablets or mini-tablets.

B. Intermediate Coating

Intermediate Coating Suspension

145 g of hydroxypropyl methylcellulose NF 6 cps is dispersed in 1.9 kg of purified water. 22.5 g esomeprazole magnesium is added to the hydroxypropyl methylcellulose solution and stirred. 290 g of magnesium stearate are dispersed in 1.3 kg of ethanol 96%. The intermediate coating suspension is sprayed onto 1.1 kg of cores from the previous step. The cores are then dried, and replaced into the fluidized bed apparatus for further coating.

C. Enteric Coating

Enteric Coating Dispersion 50.4 g of talc extra fine, 6.3 g of titanium dioxide and 12.83 g of triethyl citrate are dispersed in 600 g of purified water. The homogenized dispersion is added to 422.3 g of methacrylic acid copolymer dispersion and stirred.

The enteric coating dispersion is sprayed onto 555 g of cores from the previous step.

### Example 5

A. Drug Layer

Drug Layer Coating Suspension

180 g of hydroxypropyl methylcellulose NF 6 cps was dispersed in 3.8 kg of purified water. 180 g magnesium carbonate was added and the solution is stirred. 240 g esomeprazole magnesium was added and stirred until a homogeneous suspension was obtained.

720 g Cellets® (microcrystalline cellulose spheres) (500-710 micron) were introduced into a fluid bed apparatus and the aforementioned suspension was sprayed onto the spheres. Then the spheres were dried, sifted through a 18 mesh screen and are replaced into the fluidized bed apparatus for further coating.

B. Intermediate Coating

Intermediate Coating Suspension

145 g of hydroxypropyl methylcellulose NF 6 cps was dispersed in 1.9 kg of purified water. 22.5 g esomeprazole magnesium was added to the hydroxypropyl methylcellulose solution and stirred. 290 g of magnesium stearate were dispersed in 1.3 kg of ethanol 96%. The intermediate coating suspension was sprayed onto 1.1 kg of drug layered pellets from the previous step. The spheres were then dried, sifted through a 18 mesh screen and replaced into the fluidized bed apparatus for further coating.

C. Enteric Coating

Enteric Coating Dispersion

36 g of talc extra fine, 4.5 g of titanium dioxide and 9.2 g of triethyl citrate were dispersed in 535 g of acetone-isopropyl alcohol mixture (3:2). 90.5 g of methacrylic acid copolymer powder was dissolved in 1150 g acetone-isopropyl alcohol mixture (3:2) The homogenized dispersion was added to the methacrylic acid copolymer solution and stirred.

The enteric coating dispersion was sprayed onto 700.9 g of spheres from the previous step. The spheres were then dried, sifted through a 16 mesh screen and filled into hard gelatin capsules.

### Example 6

A. Drug Layer

Drug Layer Coating Suspension

180 g of hydroxypropyl methylcellulose NF 6 cps was dispersed in 3.8 kg of purified water. 180 g magnesium carbonate was added and the solution was stirred. 240 g esomeprazole magnesium was added and stirred until a homogeneous suspension was obtained.

720 g Cellets® (microcrystalline cellulose spheres) (500-710 micron) were introduced into a fluid bed apparatus and the aforementioned suspension was sprayed onto the spheres. Then the spheres were dried, sifted through a 18 mesh screen and were replaced into the fluidized bed apparatus for further coating.

B. Intermediate Coating

Intermediate Coating Suspension

39.2 g of hydroxypropyl methylcellulose NF 6 cps was dispersed in 0.6 kg of purified water. 12.2 g esomeprazole magnesium was added to hydroxypropyl methylcellulose solution and stirred. 54 g of ethylcellulose 7 cps were dispersed in 950 g ethanol 96%. 156.6 g of magnesium stearate were dispersed in 708 g of ethanol 96% added to the ethylcellulose dispersion and mixed for 30 minutes. The intermediate coating suspension was sprayed onto 579 g of drug layered pellets from the previous step. The spheres were then dried, sifted through a 18 mesh screen and replaced into the fluidized bed apparatus for further coating.

C. Enteric Coating

Enteric Coating Dispersion

36.5 g of talc extra fine, 4.9 g of titanium dioxide and 9.5 of triethyl citrate were dispersed in 355 g of acetone-isopropyl alcohol mixture (3:2). 91.8 g methacrylic acid copolymer powder was dissolved in 765 g acetone-isopropyl alcohol mixture (3:2). The homogenized dispersion was added to the methacrylic acid copolymer solution and stirred.

The enteric coating dispersion was sprayed onto 713.3 g of spheres from the previous step. The spheres were then dried, sifted through a 16 mesh screen and filled into hard gelatin capsules.

### Example 7

A. Drug Layer

Drug Layer Coating Suspension

210 g of hydroxypropyl cellulose NF (Klucel LF®) was dispersed in 6.4 kg of purified water. 140 g magnesium carbonate was added and the solution was stirred. 280 g esomeprazole magnesium was added and stirred until a homogeneous suspension was obtained.

840 g Cellets® (microcrystalline cellulose spheres) (500-710 micron) were introduced into a fluid bed apparatus and the aforementioned suspension was sprayed onto the spheres. Then the spheres were dried, sifted through a 18 mesh screen and were replaced into the fluidized bed apparatus for further coating.

B. Intermediate Coating

Intermediate Coating Suspension

90 g of hydroxypropyl methylcellulose NF 6 cps was dispersed in 1400 g of purified water. 14 g esomeprazole magnesium was added to hydroxypropyl methylcellulose solution and stirred. 180 g of magnesium stearate were dispersed in 830 g of ethanol 96%. The intermediate coating suspension was sprayed onto 650 g of drug layered pellets from the previous step. The spheres were then dried, sifted through a 18 mesh screen and replaced into the fluidized bed apparatus for further coating.

C. Enteric Coating

Enteric Coating Dispersion

39.3 g of talc extra fine, 4.8 g of titanium dioxide and 10.1 of triethyl citrate were dispersed in 333 g of acetone-isopropyl alcohol mixture (3:2). 89.2 g methacrylic acid copolymer powder was dissolved in 690 g acetone-isopropyl alcohol mixture (3:2). The homogenized dispersion was added to the methacrylic acid copolymer solution and stirred.

The enteric coating dispersion was sprayed onto 693.5 g of spheres from the previous step. The spheres were then dried, sifted through a 16 mesh screen and filled into hard gelatin capsules.

### Example 8

A. Drug Layer

Drug Layer Coating Suspension

195 g of hydroxypropyl cellulose NF (Klucel LF®) was dispersed in 5.9 kg of purified water. 130 g magnesium carbonate was added and the solution was stirred. 260 g esomeprazole magnesium was added and stirred until a homogeneous suspension was obtained.

650 g Suglets® (sugar spheres) (400-500 micron) were introduced into a fluid bed apparatus and the aforementioned suspension was sprayed onto the spheres. Then the spheres were dried, sifted through a 18 mesh screen and were replaced into the fluidized bed apparatus for further coating.

B. Intermediate Coating

Intermediate Coating Suspension

84.1 g of hydroxypropyl methylcellulose NF 6 cps was dispersed in 1.3 kg of purified water. 13 g esomeprazole magnesium was added to hydroxypropyl methylcellulose solution and stirred. 168.2 g of magnesium stearate were dispersed in 750 g of ethanol 96%. The intermediate coating suspension was sprayed onto 551 g of drug layered pellets from the previous step. The spheres were then dried, sifted through a 18 mesh screen and replaced into the fluidized bed apparatus for further coating.

C. Enteric Coating

Enteric Coating Dispersion

29 g of talc extra fine, 3.8 g of titanium dioxide and 7.4 of triethyl citrate were dispersed in 266 g of acetone-isopropyl alcohol mixture (3:2). 72.2 g methacrylic acid copolymer powder was dissolved in 500 g acetone-isopropyl alcohol mixture (3:2) The homogenized dispersion was added to the methacrylic acid copolymer solution and stirred.

The enteric coating dispersion was sprayed onto 563 g of spheres from the previous step. The spheres were then dried, sifted through a 16 mesh screen and filled into hard gelatin capsules.

### Example 9

A. Drug Layer

Drug Layer Coating Suspension

120 g of hydroxypropyl cellulose NF (Klucel LF®)was dispersed in 2.7 kg of purified water. 60 g of hydroxypropyl methylcellulose NF 6 cps was dispersed in 2.7 kg of purified water. Mix both polymer dispersions and stir. 120 g magnesium carbonate was added and the dispersion was stirred. 240 g esomeprazole magnesium was added and stirred until a homogeneous suspension was obtained.

720 g Cellets® (microcrystalline cellulose spheres) (500-710 micron) were introduced into a fluid bed apparatus and the aforementioned suspension was sprayed onto the spheres. Then the spheres were dried, sifted through a 18 mesh screen and were replaced into the fluidized bed apparatus for further coating.

B. Intermediate Coating

Intermediate Coating Suspension

75.4 g of hydroxypropyl methylcellulose NF 6 cps was dispersed in 1.2 kg of purified water. 11.7 g esomeprazole magnesium was added to hydroxypropyl methylcellulose solution and stirred. 150.8 g of magnesium stearate were dispersed in 690 g of ethanol 96%. The intermediate coating suspension was sprayed onto 546 g of drug layered pellets from the previous step. The spheres were then dried, sifted through a 18 mesh screen and replaced into the fluidized bed apparatus for further coating.

C. Enteric Coating

Enteric Coating Dispersion

31.2 g of talc extra fine, 4 g of titanium dioxide and 8 g of triethyl citrate were dispersed in 300 g of acetone-isopropyl alcohol mixture (3:2). 77.4 g of methacrylic acid copolymer powder was dissolved in 543 g acetone-isopropyl alcohol mixture (3:2) The homogenized dispersion was added to the methacrylic acid copolymer solution and stirred.

The enteric coating dispersion was sprayed onto 603 g of spheres from the previous step. The spheres were then dried, sifted through a 16 mesh screen and filled into hard gelatin capsules.

What is claimed is:

1. A pharmaceutical formulation of an acid labile benzimidazole compound that inhibits gastric acid secretion, comprising:
a) an inert inner core;
b) a first coating on top of the inner core comprised of the benzimidazole compound and an alkaline stabilizer;
c) an intermediate coating on top of the first coating devoid of an alkaline stabilizer, wherein the intermediate coating comprises the same or different benzimidazole compound; and
d) an outer enteric layer.

2. The pharmaceutical formulation of claim 1, wherein the inner core is an inert non-pareil sugar or MCC sphere.

3. The pharmaceutical formulation of claim 1, wherein the benzimidazole compound is selected from the group consisting of esomeprazole, lansoprazole, omeprazole, pantoprazole and rabeprazole.

4. The pharmaceutical formulation of claim 3, wherein the benzimidazole compound is a salt.

5. The pharmaceutical formulation of claim 4, wherein the benzimidazole compound is esomeprazole magnesium.

6. The pharmaceutical formulation of claim 5, wherein the esomeprazole magnesium is amorphous.

7. The pharmaceutical formulation of claim 5, wherein the esomeprazole magnesium is hydrated.

8. The pharmaceutical formulation of claim 1, wherein the first coating has about 80% to about 95% (w/w) of the benzimidazole compound present in the formulation.

9. The pharmaceutical formulation of claim 8, wherein the benzimidazole compound present in the first coating is about 85% to about 95% (w/w).

10. The pharmaceutical formulation of claim 9, wherein the benzimidazole compound present in the first coating is about 90% (w/w).

11. The pharmaceutical formulation of claim 1, wherein the alkaline stabilizer is selected from the group consisting of magnesium carbonate, magnesium oxide, calcium carbonate, sodium carbonate and mixtures thereof.

12. The pharmaceutical formulation of claim 1, wherein the alkaline stabilizer is an organic base.

13. The pharmaceutical formulation of claim 12, wherein the organic base is tris(hydroxymethyl)aminomethane.

14. The pharmaceutical formulation of claim 1, wherein the intermediate coating has about 5% to about 20% (w/w) of the benzimidazole present in the formulation.

15. The pharmaceutical formulation of claim 14, wherein the intermediate coating has about 5% to about 15% (w/w) of the benzimidazole present in the formulation.

16. The pharmaceutical formulation of claim 15, wherein the intermediate coating has about 10% (w/w) of the benzimidazole present in the formulation.

17. The pharmaceutical formulation of claim 1, wherein the intermediate coating further comprise a binder.

18. The pharmaceutical formulation of claim 17, wherein the binder is selected from the group consisting of hydroxypropyl methylcellulose, hydroxypropylcellulose, polyvinyl alcohol, polyvinyl pyrrollidone, starch, methylcellulose, ethylcellulose, carboxymethyl cellulose, sucrose solution, dextrose solution.

19. The pharmaceutical formulation of claim 1, wherein the enteric coating comprises a polymer selected from the group consisting of methacrylic acid copolymer, hydroxypropyl methylcellulose phtalate and hydroxypropyl methylcellulose acetate succinate.

20. The pharmaceutical formulation of claim 19, wherein the polymer is about 45% to about 85% (w/w) of the enteric coat layer.

21. The pharmaceutical formulation of claim 1, wherein the pharmaceutical formulation is administered as a multi-particulate delivery system.

22. The pharmaceutical formulation of claim 21, wherein the multi-particulate delivery system contains particles having a mean diameter of about 400 to about 1200 microns.

23. The pharmaceutical formulation of claim 1, wherein the pharmaceutical formulation is stable under storage at 30° C. and 65% relative humidity for at least about 2 months.

24. The pharmaceutical formulation of claim 23, wherein the pharmaceutical formulation is stable under storage at 30° C. and 65% relative humidity for at least about 3 months.

25. The pharmaceutical formulation of claim 1, wherein the formulation is in the 5 form of a tablet, an ovule, a chewable tablet, a buccal tablet, a sub-lingual tablet, a granule, a pellet, a bead, or a pill.

26. The pharmaceutical formulation of claim 25, wherein the tablet comprises compressed beads.

27. The pharmaceutical formulation of claim 1, wherein the formulation is formulated for intermediate release, controlled release, extended release, delayed released, targeted release, or targeted delayed release.

28. A process of preparing the pharmaceutical formulation of claim 1, comprising the steps of:
a) coating an inert inner core with a first suspension comprising a benzimidazole compound and an alkaline stabilizer;
b) applying a coating on the coated inner core with a second suspension comprising the same or different benzimidazole compound to obtain an intermediate coating, wherein the suspension is devoid of an alkaline stabilizer; and
c) applying an enteric coating on the intermediate coating.

29. The process of claim 28, wherein the first suspension is an aqueous suspension.

30. The process of claim 28, wherein the benzimidazole compound is selected from the group consisting of esomeprazole, lansoprazole, omeprazole, pantoprazole and rabeprazole.

31. The process of claim 30, wherein the benzimidazole compound is esomeprazole magnesium.

32. The process of claim 28, wherein the enteric coating is applied from a solution in an organic solvent.

33. A pharmaceutical formulation of an acid labile benzimidazole compound that inhibits gastric acid secretion comprising:
a) an inner core comprised of a benzimidazole compound and an alkaline stabilizer;
b) an intermediate coating on said inner core devoid of an alkaline stabilizer comprising the same or different benzimidazole compound; and
c) an outer enteric layer.

34. The pharmaceutical formulation of claim 33, wherein the benzimidazole compound is selected from the group consisting of esomeprazole, lansoprazole, omeprazole, pantoprazole and rabeprazole.

35. The pharmaceutical formulation of claim 34, wherein the benzimidazole compound is a salt.

36. The pharmaceutical formulation of claim 35, wherein the benzimidazole compound is esomeprazole magnesium.

37. A process of preparing a pharmaceutical formulation of claim 33, comprising the steps of:
a) preparing an inner core by mixing a benzimidazole compound and an alkaline stabilizer;
b) layering the inner core to form an intermediate coating with a suspension comprising the benzimidazole compound, wherein the suspension is devoid of an alkaline stabilizer; and
c) layering the intermediate to form an enteric coating.

38. A multi-particulate delivery system of an acid labile benzimidazole compound that inhibits gastric acid secretion, comprising a plurality of particles comprised of:
a) an inert inner core;
b) a first coating on the inner core comprised of a benzimidazole compound and an alkaline stabilizer, wherein the benzimidazole compound is present in an amount of about 80% to about 95% (w/w) of the labeled dose of the benzimidazole compound in the formulation;
c) an intermediate coating devoid of the alkaline stabilizer on top of the first coating, wherein the intermediate coating comprises the benzimidazole compound, wherein the benzimidazole compound is present in an amount of about 5% to about 20% (w/w) of the labeled dose of benzimidazole compound in the formulation; and
d) an outer enteric layer.
